(19) United States
(12) Patent Application Publication
(10) Pub. No.: US 2016/0296181 A1
(43) Pub. Date: Oct. 13, 2016

(54) METHOD AND DEVICE FOR DENTAL IMAGING

(71) Applicant: SIRONA DENTAL SYSTEMS GMBH, Bensheim (DE)

(72) Inventors: Ciamak ABKAI, Heddesheim (DE); Kai LINDENBERG, Wersau (DE); Johannes ULRICI, Darmstadt (DE); Christian BECKHAUS, Darmstadt (DE); Bernhard SCHMITT, Ludwigshafen (DE)

(73) Assignee: DENTSPLY SIRONA Inc., York, PA (US)

(21) Appl. No.: 15/038,660

(22) PCT Filed: Nov. 24, 2014
(86) PCT No.: PCT/EP2014/075373

§ 371 Date: May 23, 2016

(30) Foreign Application Priority Data
Nov. 22, 2013 (DE) 10 2013 223 958.0

(51) Int. Cl. A61B 6/14; G06T 11/00; G06T 7/00; A61B 6/02; A61B 6/00

(52) U.S. Cl. CPC A61B 6/14; A61B 6/02; A61B 6/463; A61B 6/5258; G06T 7/0012; G06T 11/003; G06T 2207/30036

(57) ABSTRACT

The present invention relates to a method for dental imaging. The imaging method involves providing an image data record relating to at least one maxillofacial image region, and creating a representation of the at least one maxillofacial image region from the image data record, in such a way that the representation includes information relating to the quality of the image data record on which the representation is based.

### TECHNICAL FIELD

The present invention relates to a method for dental imaging. In addition, the present invention relates to a computer program that carries out all the steps of the inventive method when it is running on a computer, as well as to a data medium that stores this computer program. Finally, the invention relates to an imaging system for dental application that is designed to perform the method according to the invention.

### PRIOR ART

The quality of images in dental imaging can be impaired by a variety of causes. In the case of x-rays, these causes include, for example, metals in an object to be examined, or movements of the patient and device, which can lead to artifacts. In the case of digital volume tomography (DVT) in which a three-dimensional volume is calculated from different individual projections from different directions, additional causes of quality impairment are, among other things, projections with overexposed image areas, an insufficient number of projections to reconstruct the three-dimensional volume, an excessively small angular range covered by the projections, or truncation artifacts if the object to be examined is not completely irradiated in all the images. The quality of images from panorama x-ray machines can also be impaired by metals in an object to be examined, or movements by the patient and device, as well as by so-called antagonistic arch artifacts. Intraoral x-rays lose quality if the x-ray sensor is obliquely positioned relative to the x-ray tube, or due to movements of the patient and device. The quality of magnetic resonance tomograms is reduced by metals or movements of the patient and device. Such problems do not necessarily impair the image quality of the entire image. In the case of a DVT image, metals produce distorted images particularly in their immediate vicinity, whereas more distant image regions can be depicted almost error-free. Swallowing movements on the part of the patient yield image distortions, particularly in the vicinity of the tongue and palate. Another example of a factor that only influences the image quality of part of an image is the angular range covered by the projection for image reconstruction. Whereas projections comprising an angular range necessary for an accurate reconstruction exist at the center of the object, projections from a restricted angular range may only be available at the edge regions of the object, so that the image quality at that location is therefore restricted. Poor image quality can prevent the image from being accurately evaluated by the physician. This can lead to misdiagnoses or even render the image useless which, in the case of x-ray imaging, causes the patient to be unnecessarily re-exposed to radiation. It would be desirable for the diagnosing physician to possess additional information on whether or not the diagnosis-relevant parts of an image are subject to artifacts, or on the quality level of the image at the location to be diagnosed. The physician would thereby be supported in the assessment of whether anomalies are produced in the image by artifacts or medical causes. This could reduce the risk of misdiagnoses as well as unnecessary repeat images. In addition, if suboptimal image quality occurs repeatedly, the physician is provided with an indicator of whether adaptations are necessary in the treatment process, such as in positioning the patient, or whether equipment faults exist, such as calibration errors. It is therefore the object of the present invention to provide a method that assists a physician in determining whether image defects are caused by artifacts or are ascribable to medical causes, so that this determination by the physician no longer has to be exclusively made based on his image diagnosing experience.

### DESCRIPTION OF THE INVENTION

This object is achieved by the method according to the invention for dental imaging that comprises the following steps: The provision of an image data record of at least one maxillofacial image region, in particular of a jaw or jaw section, and the creation of a representation of the at least one maxillofacial image region from the image data record, wherein information on the quality of the image data record on which the representation is based is integrated into the representation. The image data record is acquired with an acquisition device. According to the invention, an “image data record” is understood to be both a uniform image data record as well as an image data record composed of a plurality of partial data records. If an image data record consists of a plurality of partial data records, a distortion of the image quality can occur in particular in an overlapping region of the partial data records which can be visualized by the method according to the invention. The image data record is in particular a two-dimensional, three-dimensional or four-dimensional image data record, wherein the latter can for example be realized by integrating a time axis into the image data record. Preferably, it is a three-dimensional image data record that in particular can be acquired by a DVT device as the acquisition device. Data on the quality of the data points of the image data record can be determined before or after creating the representation of the at least one maxillofacial image region. Data on the quality of the image data record is obtained by an analysis of the image data record, or by an analysis of the parameters of the acquisition of the image data record, in particular an algorithmic analysis. This analysis can, for example, be used to identify movements of the patient, or losses in quality arising from the presence of highly absorbent metals in a region to be examined. Furthermore, this analysis can be used to identify losses of quality intrinsic to the method, such as truncation artifacts, or losses in quality ascribable to the image geometry. In another preferred embodiment of the invention, the data on the quality of the image data record is acquired by an analysis of the imaging method used. Losses in quality, such as noise or cone artifacts, can thereby be identified that are ascribable to basic physical restrictions in the imaging and that, for example, result from the image geometry, from the used path of the acquisition device around the patient, or from imaging parameters, such as kV/mA settings. In another preferred embodiment of the invention, the data on the quality of the image data record is obtained through an analysis of data from at least one sensor that monitors the acquisition of the image data record. This sensor can, for example, be a camera that observes a movement of the patient parallel to an x-ray being taken for acquiring the image data record. Information on the quality of the image data record on which the representation is based relates in particular to its image sharpness, its freedom from artifacts, and/or the precision of positions or measuring data. Preferably, a user of the method according to the invention is provided with a way to only display certain quality information and/or specify thresholds for the display of the quality information. Information on the global quality of the image data record on which the representation is based can be integrated into the representation of the at least one maxillofacial image region. In addition or alternatively, information on the local quality of the image data record on which the representation is based can be integrated into the representation. According to the invention, “local quality” is understood to be the quality of a partial region of the image data record on which the representation is based. In one preferred embodiment of the invention, the information on the global and/or local quality of the image data record on which the representation is based is visualized by a color code. In doing so, the global quality of the image data record can, for example, be visualized by a stoplight. The local display of the image quality can in particular be realized by a semitransparent coloration of the image, wherein the image regions with different quality are colored with different colors. It is also possible according to the invention to encode the local image quality by the color intensity. Alternatively, as opposed to a coloration, the image quality can be displayed locally in that a stoplight displays the image quality only at a location selected by the user. This selection can in particular be made by the user moving a cursor, a magnifying glass, or an examination window of a computer program to a place in the representation at which information on the local quality of the underlying image data record is to be visualized. Alternatively to showing a color with a stoplight, the edge of the examination window, or the magnifying glass, or the cursor can be colored following this selection. In another preferred embodiment of the invention, the information on the global and/or local quality of the representation underlying the image data record can be visualized by presenting a quality index. The quality index can for example be locally visualized by displaying the quality index when a cursor passes over a region of the image. In yet another preferred embodiment of the invention, information on the global and/or local quality of the image data record on which the representation is based can be topologically visualized (like a mountain range), i.e., local differences in quality are symbolized by contour lines. The quality of the image data record or the presence of a flaw can depend on the direction if, for example, an image point is blurred in one direction of movement of the patient, and not in another direction in space. In order to take this directional dependency into account in the display of image quality, it is preferable according to the invention to integrate information on the direction-dependent quality of the image data record on which the representation is based into the representation of the at least one maxillofacial image region. In this context, the image quality information is no longer one-dimensional, each pixel or image region having just one precisely assigned image quality value, but is instead multidimensional, so that the pixels or image regions accordingly possess different image quality values depending on the direction. The information on the direction-dependent quality of the image data record on which the representation is based is visualized in particular by a vector field. In this vector field, the direction of the arrow of the vectors can describe the direction of a flaw, and the arrow length can describe a measure of the loss of quality. It is possible according to the invention for the information on the quality of the image data record on which the representation is based to not be displayed permanently in the representation. In one preferred embodiment of the invention, the display only occurs in response to a user interaction. This can for example be the movement of a cursor such that the position of the cursor determines the point of the representation for which quality information is displayed. In another embodiment of the invention, the information on the quality of the image data record on which the representation is based is displayed depending on a software application used by a physician. If, for example, the physician wishes to measure a length in the representation, the displayed information on the quality can consist of the imprecision of a position or measurement, such as in the form of a confidence interval. In yet another embodiment of the invention, the display of information on the quality of the representation can be manually turned on and off. Furthermore, it is possible according to the invention to also provide an isolated representation of the quality information in the representation of the at least one maxillofacial image region, in addition to integrating the information on the quality of the image data record on which the representation is based. It is preferable to assign a representation of information about a cause of a quality reduction of the image data record to at least one representation of information on the quality of the image data record on which the representation is based. This representation of information about the cause of the quality reduction can, for example, be provided by a text or symbol. It is particularly preferable to assign to at least one cause of a quality reduction a measure that, upon being introduced, entirely or partially compensates for the reduction in quality. Such a measure can, for example, consist of the use of a correction algorithm. It is most preferable for this measure to be able to be initiated by a user as an additional step of the method according to the invention. Furthermore, it is most preferable for the quality improvement achievable or achieved by introducing this measure to also be visualized. The invention furthermore relates to a computer program that performs all the steps of the method according to the invention when it is run on a computer. This makes it possible to implement the method according to the invention in available data processing devices for dental imaging without having to perform structural modifications of said devices. The computer program according to the invention can be saved on a data medium. By downloading the computer program according to the invention onto an imaging system for the dental application, an imaging system can be obtained that is configured to perform the method according to the invention. In particular, the imaging system can be a two-dimensional or three-dimensional system. The system is preferably three-dimensional, wherein a digital volume tomography system is most preferred.

### BRIEF DESCRIPTION OF THE DRAWINGS

Exemplary embodiments of the invention are illustrated in the drawings and explained in greater detail in the following description.

FIG. 1 shows the acquisition of an image data record in one embodiment of the method according to the invention.

FIG. 2 shows a representation of two jaws in a method according to one embodiment of the invention.

FIG. 3 shows a representation of a jaw in a second embodiment of the invention.

FIG. 4 shows a representation of a jaw in a third embodiment of the invention.

FIG. 5 shows a representation of a jaw in a fourth embodiment of the invention.

### EXEMPLARY EMBODIMENTS

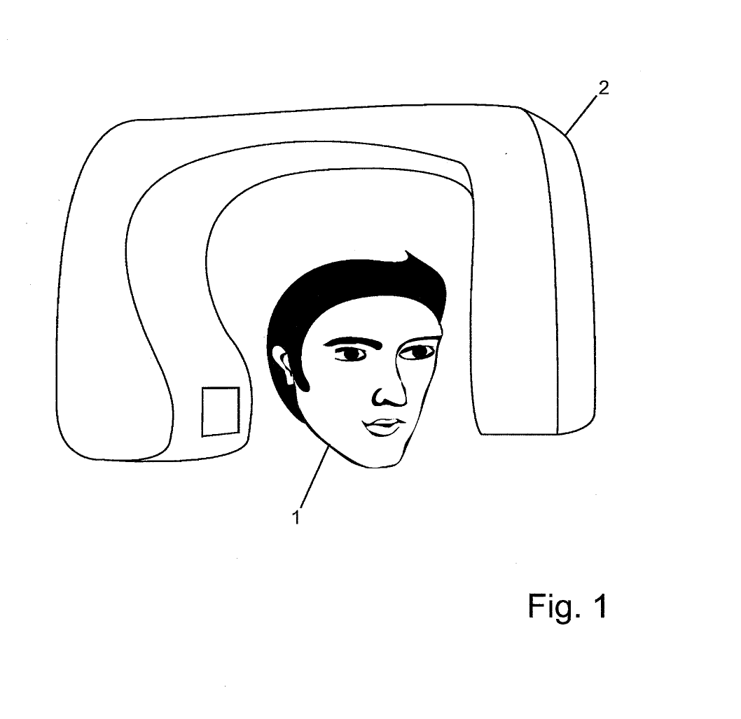

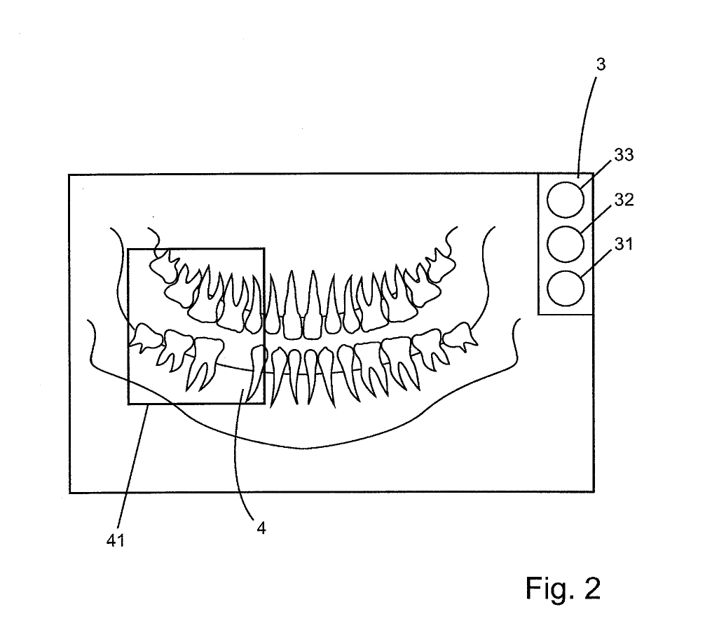

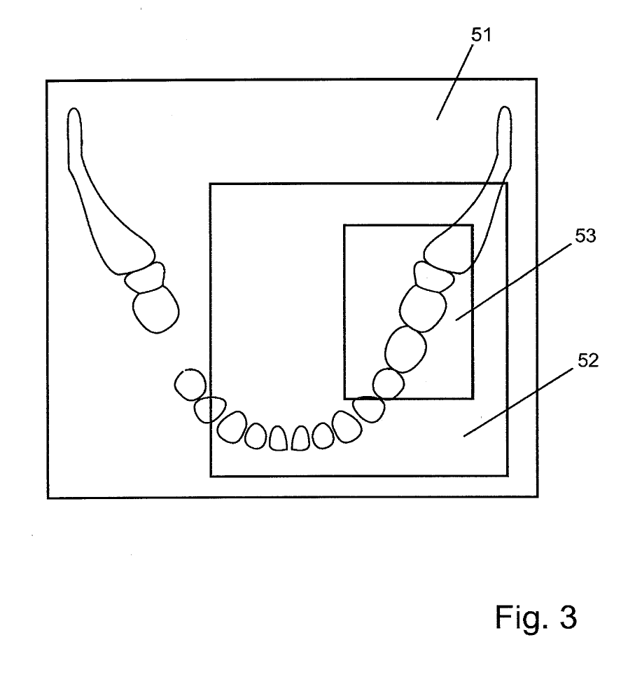

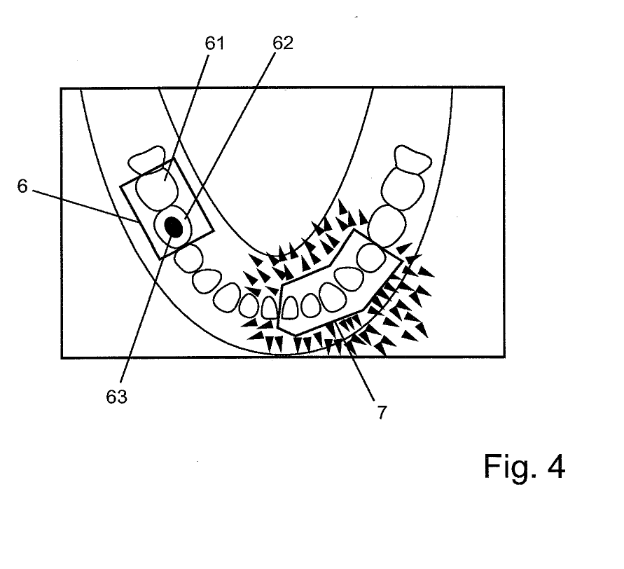

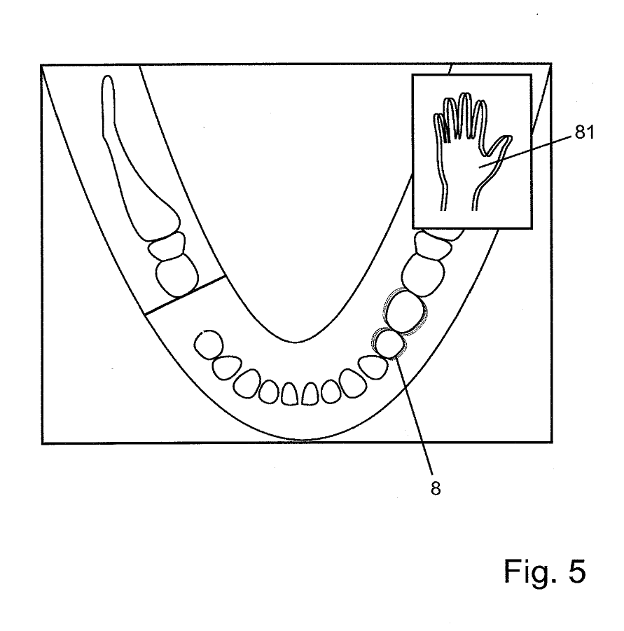

In an embodiment of the method for dental imaging according to the invention shown in FIG. 1, a three-dimensional image data record of two jaws of a patient 1 is acquired by a DVT system. Data on the quality of the data points of the image data record are acquired, and then a representation of the two jaws is prepared by means of a computer program. This is shown in FIG. 2. Information on the global quality of the image data record on which the representation is based is displayed by a stoplight 3. A first color 31, such as green, shows that the image data record corresponds to the anticipated image quality. A second color 32, such as yellow, indicates image distortions deviating from the norm. A third color 33, such as red, indicates strong image distortions. In the latter case, diagnostic conclusions should only be drawn with caution. A local display of the image quality can be triggered by a physician by positioning an examination window 4 over a section of the representation to be examined. The frame 41 of this examination window then assumes one of the three colors 31, 32, 33 according to the image quality of the region examined. In another embodiment of the method according to the invention, image regions with a different quality of the underlying image data record are color-coded by coloring the image regions with different colors. FIG. 3 shows a representation generated according to this embodiment of the method with three image regions 51, 52, 53, wherein, for example, the image region 51 has the anticipated image quality and is colored green, the image region 52 has image distortions deviating from the norm and is colored yellow, and the image region 53 has strong image distortions and is colored red. In a method according to another embodiment of the invention, information is visualized on directionally independent and directionally dependent quality of the image data record on which the representation is based. As in the previous embodiment of the invention, directionally independent information on the image quality is visualized by displaying different regions of the representation in different colors. This is shown in FIG. 4. Accordingly, image quality changes resulting from a metal artifact, such as an amalgam filling, are encoded in a region 6 by the different colors 61, 62, 63. Directionally dependent changes in the quality of the image data record arising from a movement of the patient are visualized in another region of the jaw by a vector field 7. In this case, the direction of the vector arrows indicates the direction of the change in quality, and the arrow length indicates the magnitude of the change in quality. In yet another embodiment of the method according to the invention, the cause of impairments of the quality of the image data record on which the representation is based is visualized by symbols. This is shown in FIG. 5. If the physician selects an image region 8 in which an impairment of quality has occurred due to a movement, this is for example indicated by a symbol 81, a shaky hand. By clicking or moving a cursor onto the symbol, additional information and proposals for measures appear that can be selected to completely or partially compensate for the reduction in quality. This can be, for example, the use of a correction algorithm. The improvement in quality that is achievable by initiating the measure can also be visualized. All of the above-described embodiments of the method according to the invention can be implemented as a computer program on a digital volume tomography system 2 to help the physician determine whether image defects in a DVT representation are caused by artifacts or are ascribable to medical causes.

1. A method for dental imaging, comprising the steps of:
acquiring an image data record of at least one maxillofacial image region by means of an acquisition device, and
obtaining data on the quality of the image data record by an analysis of the image data record, or by an analysis of the parameters of the acquisition of the image data record, and
preparing a representation of the at least one maxillofacial image region from the image data record, wherein information on the quality of the image data record is incorporated into the representation.

2. The method according to claim 1, wherein the data on the quality of the image data record is obtained through an analysis of data from at least one sensor that monitors the acquisition of the image data record.

3. The method according to claim 1, further comprising the step of integrating information on the global quality and/or local quality of the image data record on which the representation is based into the representation.

4. The method according to claim 3, wherein information on the global and/or local quality of the image data record on which the representation is based is visualized by color coding.

5. The method according to claim 1, further comprising the step of integrating information on the directionally dependent quality of the image data record on which the representation is based into the representation.

6. The method according to claim 5, wherein the information on the directionally dependent quality of the image data record on which the representation is based is visualized by a vector field.

7. The method according to claim 1, further comprising the step of assigning a representation of information about a cause of a quality reduction of the image data record to at least one representation of information on the quality of the image data record on which the representation is based.

8. The method according to claim 7, further comprising the step of assigning at least one measure to at least one cause of a reduction in quality that, upon being introduced, entirely or partially compensates for the reduction in quality.

9. The method according to claim 8, wherein a user interaction makes possible the introduction of the measure.

10. The method according to claim 9, wherein the quality improvement achievable by the introduction of the measure is also visualized.

11. A computer program that carries out all the steps of a method according to claim 1 when it is run on a computer.

12. A data medium, wherein it stores a computer program according to claim 11.

13. An imaging system for dental application, wherein it is designed to execute a method according to claim 1.

14. The imaging system according to claim 13, wherein it is a three-dimensional imaging system.

15. The imaging system according to claim 14, wherein it is a digital volume tomography system.
